(19) United States
(12) Patent Application Publication
(10) Pub. No.: US 2005/0251062 A1
(43) Pub. Date: Nov. 10, 2005

(54) Iterative approach for applying multiple currents to a body using voltage sources in electrical impedance tomography

(76) Inventors: Myoung Choi, Seoul (KR); David Isaacson, Latham, NY (US); Jonathan C. Newell, Glenmount, NY (US)

Correspondence Address: NOTARO AND MICHALOS, 100 DUTCH HILL ROAD, SUITE 110, ORANGEBURG, NY, 10962-2100, US

(21) Appl. No.: 11/124,246

(22) Filed: May 6, 2005

Related U.S. Application Data
(60) Provisional application No. 60/569,549, filed on May 10, 2004.

(51) Int. Cl. A61B 5/05

(52) U.S. Cl. 600/547

(57) ABSTRACT

Voltage sources produce desired current patterns in an ACT-type Electrical Impedance Tomography (EIT) system. An iterative adaptive algorithm generates the necessary voltage pattern that will result in the desired current pattern. The convergence of the algorithm is shown under the condition that the estimation error of the linear mapping from voltage to current is small. The simulation results are presented along with the implication of the convergence condition.

### CROSS-REFERENCE TO RELATED APPLICATION

This U.S. patent application claims priority on, and all benefits available from U.S. provisional patent application No. 60/569,549 filed May 10, 2004, all of which is incorporated here by reference.

### STATEMENT OF GOVERNMENT INTEREST

Development of the present invention was supported, in part, by CenSSIS, the Center for Subsurface Sensing and Imaging Systems, under the Engineering Research Center Program of the National Science Foundation (Award number EEC-9986821).

### FIELD AND BACKGROUND OF THE INVENTION

The present invention relates generally to the field of EIT, and in particular to a new and useful appartaus and method for Adaptive Current Tomography (ACT).

Electrical Impedance Tomography (EIT) is a technique for determining the electrical conductivity and permittivity distribution within the interior of a body from measurements made on its surface. Typically, currents are applied through electrodes placed on the body's surface and the resulting voltages are measured. Alternately, voltages can be applied and the resulting currents are measured. Recent reports on a number of EIT systems can be found in: [3] R. D. Cook, G. J. Saulnier, D. G. Gisser, J. C. Goble, J. C. Newell, and D. Isaacson, “ACT 3: A high speed, high precision electrical impedance tomography,” IEEE Trans. on Biomedical Eng., vol.41, pp.713-722, August 1994; [4] R. W. M. Smith, I. L. Freeston, and B. H. Brown, “A real-time electrical impedance tomography system for clinical use—Design and preliminary results,” IEEE Trans. on Biomedical Eng., vol.42, pp.133-140, February 1995; [5] P. M. Edic, G. J. Saulnier, J. C. Newell, D. Isaacson, “A real-time electrical impedance tomograph,” IEEE Trans. on Biomedical Eng., vol.42, no.9, pp.849-859, September 1995; [6] P. Metherall, D. C. Barber, R. H. Smallwood, and B. H. Brown, “Three-dimensional electrical impedance tomography,” Nature, vol.380, pp.509-512, April 1996; and [7] A. Hartov, R. A. Mazzarese. F. R. Reiss, T. E. Kerner, K. S. Osterman, D. B. Williams, and K. D. Paulsen, “A multichannel continuously selectable multifrequency electrical impedance spectroscopy measurement system,” IEEE Trans. on Biomedical Eng., vol.47, no.1, pp.49-58, January 2000.

Some systems apply currents to a pair of adjacent electrodes, with the current entering at one electrode and leaving at another, and measure voltages on the remaining electrodes. In these Applied Potential Tomography (APT) systems, the current is applied to different pairs of electrodes, sequentially to produce enough data for an image. In Adaptive Current Tomography (ACT) systems, currents are applied to all the electrodes simultaneously and multiple patterns of currents are applied to produce the data necessary for an image. If the body being imaged is circular or cylindrical and measurements are performed using a single ring of electrodes around the body, the most common current patterns are spatial sinusoids of various frequencies. In this invention, we focus on a current delivery system for an ACT-type EIT system that uses voltage sources.

The image reconstruction problem in EIT is ill-posed, and large changes in the conductivity and permittivity in the interior can produce small changes in the currents or voltages at the surface. As a result, measurement precision in EIT systems is of critical importance. It is known that when current is applied and the resulting voltages are measured, the errors in the measured data are reduced as the spatial frequency increases, proportional to the inverse of the spatial frequency. Conversely, the error is amplified in proportion to the spatial frequency when a voltage distribution is applied and the resulting current is measured. See [1] D. Isaacson, “Distinguishability of conductivities by electric current computed tomography”, IEEE Trans. on Medical Imaging, Ml-5(2):92-95, 1986. Hence, the current source mode is superior to the voltage mode in terms of the high frequency noise suppression and higher accuracy in the conductivity image.

In practice, however, current sources are difficult as well as expensive to build. See [2] A. S. Ross, An Adaptive Current Tomograph for Breast Cancer Detection. Ph.D. Thesis, Rensselaer Polytechnic Institute, Troy, N.Y., 2003. Building a high precision current source is a technologically challenging task. The current source must have output impedance sufficiently large compared to the load, at the operating signal frequency to ensure that the desired current is applied for various loads. It is even more difficult to design a current source if the EIT system is to operate over a wide range of signal frequencies, as is required for EIT spectroscopy. The implementation of high-precision current sources has generally required the use of calibration and trimming circuits to adjust output impedance up to sufficient levels, yielding relatively complex circuits.

A voltage source, however, is easier and less expensive to build and operate compared to a current source. It requires smaller circuit board space, and can be easily and quickly calibrated. EIT systems using voltage sources have been implemented, though these systems suffer from increased sensitivity to the high frequency noise described above. Ideally, one would like the simplicity of voltage sources with the noise advantages of current sources.

The approach of the present invention uses voltage sources to produce the desired current pattern in an ACT-type EIT system. The amplitude and phase of a voltage source need to be adjusted in a way that produces the desired current.

An iterative algorithm was reported in [8] A. Hartov, E. Demidenko, N. Soni, M. Markova, and K. Paulsen, “Using voltage sources as current drivers for electrical impedance tomography”, Measurement Science and Technology, vol. 13, pp. 1425-1430, 2002, where the individual voltage sources were adjusted using a concept of an effective load, and the current was shown to converge to the desired value in a majority of the experiments. According to the present invention, a computation algorithm that generates the voltages in a more systematic way is disclosed, and the condition of the current convergence is given in an explicit form.

At present, an EIT system at Rensselaer Polytechnic Institute is ACT 3, which uses current sources only. The next version of EIT system under development is ACT 4 and it has voltage as well as the current sources. The present invention in meant to replace the high precision current source by generating the current by software using a voltage source.

### SUMMARY OF THE INVENTION

It is an object of the present invention to provide a method for using voltage sources to produce a desired current pattern in an EIT system.

It is a further object of the present invention to provide an iterative adaptive algorithm set for generating the necessary voltage pattern that will result in the desired current pattern.

Accordingly, an EIT method is provided for determining an electrical conductivity and an electrical permittivity distribution within a body from measurements made at a plurality of electrodes spaced on a surface of the body. The method begins by providing a plurality of voltage sources for producing a plurality of voltage patterns that are each calculated using an iterative calculation process.

The calculation process involves selecting a desired current vector (I^{d}) and an error tolerance (ε), using a first algorithm to compute an orthonormal basis set, and using a second algorithm with the orthonormal basis set and the desired current vector to compute an estimate of a non-singular linear mapping matrix for converting coordinate vector for voltage vector with respect to the orthonormal basis set to coordinate vector for current vector with respect to the orthonormal basis set, and to compute coordinate vector for the desired current vector (I^{d}).

A third algorithm includes computing and applying to the electrodes, the voltages of the voltage vector as a function of the estimate of the non-singular linear mapping matrix and the coordinate vector for the desired current vector. The resulting current vector is measured. The coordinate vector is computed for the measured resulting current vector with respect to the orthonormal basis set. The last part of this third algorithm involves calculating a norm of the actual error between the coordinate vector for the measured resulting current vector and the coordinate vector for the desired current vector. If the norm of the actual error is less than the selected error tolerance, the computed voltage vector of the third algorithm is used in a plurality of voltage sources to create voltage patterns, which are applied to the electrodes of an EIT system to create resulting current patterns in the body. The resulting current patterns are measured at the electrodes to determine the conductivity and permittivity distributions within the body.

If the norm of the actual error is greater than the selected error tolerance, then the third algorithm is repeated.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

FIG. 1 is a flowchart illustrating a first algorithm used according to the present invention;

FIG. 2 is a flowchart illustrating a second algorithm used according to the present invention;

FIG. 3 is a flowchart of the calculation method of the invention involving a first, a second, and a third algorithm of the present invention;

FIG. 4 is a schematic circuit diagram of a voltage source according to the invention;

FIG. 5 is a set of graphs showing convergence of the current output when no current measurement noise is present, the X axis represents iteration counts;

FIG. 6 is a set of graphs showing convergence of the current output when current measurement noise is present, the X axis representing iteration counts; and

FIG. 7 is a set of correlated graphs showing variation of the absolute value for Q or ∥Q∥, which is one, minus the ratio between an estimate (Â) for a nonsingular linear matrix (A) and the matrix itself, that is Q=(1−Â/A), where I=AV, I being the measured current which equals A times the applied voltage V, plotting iteration counts on the x-axes and Q norm on the y-axes;

### DESCRIPTION OF THE PREFERRED EMBODIMENTS

For the purpose of explaining the present invention, let I=(I₁, I₂, . . . Iₗ)^{T} denote an L×1 electrode current vector where Iₙ is the current value on electrode n, and L is the number of electrodes. Similarly, let V=(V₁, V₂, . . . Vₗ)^{T} denote an L×1 electrode voltage vector. The mapping from the applied electrode voltage V to the measured electrode current I can be represented using a constant L×L matrix A, so that I=AV, provided that the change with time in the electrical conductivity of human body under examination is assumed to be negligible or the change is slow compared to the fast sampling time of the measurement data. Since the magnitude and the phase of the currents and voltages are used in the conductivity and permittivity reconstruction, the elements of I, V and A are complex numbers.

The goal is to compute voltage V^{d} that will generate the desired electrode current pattern I^{d}. The exact value of A can not be determined. The estimate of A, denoted as Â, can be obtained experimentally by applying a set of independent current patterns and measuring the corresponding output voltages. Then, Â can be used to compute V^{d}. However, Â would contain errors due to modeling errors in the geometry of the electrodes in addition to the measurement errors.

According to the present invention, an iterative algorithm for computing the voltage V^{d}=(V₁^{d}, V₂^{d}, . . . V_{L}^{d})^{T} is presented that will produce a desired current pattern I^{d} with high precision in the presence of the estimation errors in Â.

Consider the following exemplary algorithm:

Given a nonsingular estimate Â of the linear mapping A from voltage to current, I=AV, a desired current I^{d}, and error tolerance ε, find the voltage V* that will produce I*=AV* such that ∥e∥=∥I^{d}−I*∥<ε.
1. eₒ=I^{d}, V⁰=0, k=0
2. k=k+1, Compute V^{k}=V^{k-1}+Â⁻¹eₖ₋₁ Apply V^{k}, and measure I^{k}, Compute eₖ=I^{d}−I^{k}.
3. If ∥eₖ∥<ε then V*=V^{k} and stop, Else go to 2

Theorem 1. The k-th error in the exemplary algorithm is eₖ=Q^{k}I^{d} where Q=(1−AÂ⁻¹). Furthermore, if ∥Q∥<1, then ∥eₖ∥<∥eₖ₋₁∥ and ∥eₖ∥<∥Q∥^{k}∥e₀∥ hold for k≧1.

(pf) Let us suppose the assumption is true for (k-1)th step, i.e. eₖ₋₁=Q^{k-1}I^{d}=(I−AÂ⁻¹)^{k-1}I^{d}.

Then, V^{k}=V^{k-1}+Â⁻¹eₖ₋₁=V^{k-1}+Â⁻¹(I−AÂ⁻¹)^{k-1}I^{d}

Also,

$$\begin{matrix}{I}^{k} = {AV}^{k} \\ = {AV}^{k - 1} + A {{\hat{A}}^{- 1} ( I - A {\hat{A}}^{- 1} )}^{k - 1} {I}^{d} \\ = {I}^{k - 1} + A {{\hat{A}}^{- 1} ( I - A {\hat{A}}^{- 1} )}^{k - 1} {I}^{d}\end{matrix}$$

The error at k-th step is

$$\begin{matrix}{e}_{k} = {I}^{d} - {I}^{k} \\ = {I}^{d} - {I}^{k - 1} - A {{\hat{A}}^{- 1} ( I - A {\hat{A}}^{- 1} )}^{k - 1} {I}^{d} \\ = {e}_{k - 1} - A {{\hat{A}}^{- 1} ( I - A {\hat{A}}^{- 1} )}^{k - 1} {I}^{d} \\ = ( I - A {\hat{A}}^{- 1} ) {( I - A {\hat{A}}^{- 1} )}^{k - 1} {I}^{d} \\ = {( I - A {\hat{A}}^{- 1} )}^{k} {I}^{d} \\ = {Q}^{k} {I}^{d}\end{matrix}$$

The above is true for k=1, i.e.
e₁=I^{d}−I¹=I^{d}−AÂ⁻¹I^{d}=(I−AÂ⁻¹)I^{d}=QI^{d}
Thus, the error expression is proved. Next, the convergence of the error is shown.
∥eₖ∥=∥Q^{k}I^{d}∥=∥Qeₖ₋₁∥≦∥Q∥∥eₖ₋₁∥<∥eₖ₋₁∥
Also, ∥eₖ∥≦∥Q∥∥eₖ₋₁∥≦∥Q∥²∥eₖ₋₂∥≦ . . . ≦∥Q∥^{k}∥e₀∥
Since ∥Q∥<1 by assumption, we have ∥eₖ∥<∥Q∥^{k}∥e₀∥

Theorem 1 requires the nonsingularity of Â as well as the bound on the estimation error of Â in the form of ∥Q∥<1. When the voltage pattern is applied and a current pattern is produced, the sum of the electrode currents through the body is zero. Because of this constraint on the electrode current values, the dimension of the current vector space is L-1, while the dimension of the voltage space is L. The linear mapping A from the voltage space to the current space given by I=AV is a singular mapping and it can not be used in Theorem 1 directly.

The linear mapping from voltage space to the current space can be formulated as a nonsingular mapping if the sum of the applied electrode voltages is constrained to be zero. Then, the dimensions of the voltage subspace and current subspace are both L-1, and the mapping from L-1 dimensional voltage subspace to L-1 dimensional current subspace can be represented by a (L-1)×(L-1) nonsingular matrix. The orthonormal basis set

$${\{ {T}^{n} \}}_{n = 1}^{L - 1}$$

is chosen for the voltage and current subspaces, such that

$$\begin{matrix}{\sum }_{n = 1}^{L} {I}_{n} = {\sum }_{n = 1}^{L} {V}_{n} = 0 , {T}^{n} = {[ \begin{matrix}{T}_{1}^{n} & {T}_{2}^{n} & ⋯ & {T}_{L}^{n}\end{matrix} ]}^{T} , 〈 {T}^{k} , {T}^{x} 〉 = {δ}_{k , x} , \\ {\sum }_{n = 1}^{L} {T}_{n}^{k} = 0 ,\end{matrix}$$

where <T^{k},T^{x}> is the inner product of T^{k} with T^{x}. The current and voltage vectors can be represented as coordinate vectors with respect to the basis vector set.

$$I = {\sum }_{n = 1}^{L - 1} {i}_{n} {T}^{n} ,$$

where iₙ=<I,Tⁿ>

$$V = {\sum }_{n = 1}^{L - 1} {v}_{n} {T}^{n} ,$$

where νₙ=<V,Tⁿ>
In the above expression, iₙ and νₙ are the n-th coordinates of the current I and voltage V with respect to the basis Tⁿ. Apply voltage T^{k} and measure I^{k}, K=1,2 . . . L-1. Then, I^{k}=AT^{k}. The relationship from the applied voltage V to the measured current I is,

$$\begin{matrix}I = AV \\ {\sum }_{m = 1}^{L - 1} {i}_{m} {T}^{m} = {\sum }_{n = 1}^{L - 1} {v}_{n} A {T}^{n} \\ {\sum }_{m = 1}^{L - 1} {i}_{m} {T}^{m} = {\sum }_{n = 1}^{L - 1} {v}_{n} {I}^{n}\end{matrix}$$

Taking the inner product of both sides with T^{u},u=1,2, . . . , L-1

$$\begin{matrix}\begin{matrix}{i}_{u} = {\sum }_{n = 1}^{L - 1} 〈 {T}^{u} , {I}^{n} 〉 {v}_{n} , & u = 1 , 2 , ⋯ , L - 1\end{matrix} \\ \begin{matrix}Let & i = {[ \begin{matrix}{i}_{1} & {i}_{2} & ⋯ & {i}_{L - 1}\end{matrix} ]}^{T} , & v = {[ \begin{matrix}{v}_{1} & {v}_{2} & ⋯ & {v}_{L - 1}\end{matrix} ]}^{T} & then ,\end{matrix} \\ i = [ \begin{matrix}{i}_{1} \\ {i}_{2} \\ ⋮ \\ {i}_{L - 1}\end{matrix} ] = [ \begin{matrix}〈 {T}^{1} , {I}^{1} 〉 & 〈 {T}^{1} , {I}^{2} 〉 & ⋯ & 〈 {T}^{1} , {I}^{L - 1} 〉 \\ 〈 {T}^{2} , {I}^{1} 〉 & 〈 {T}^{2} , {I}^{2} 〉 & ⋯ & 〈 {T}^{2} , {I}^{L - 1} 〉 \\ ⋮ & ⋮ & ⋮ & ⋮ \\ 〈 {T}^{L - 1} , {I}^{1} 〉 & 〈 {T}^{L - 1} , {I}^{2} 〉 & ⋯ & 〈 {T}^{L - 1} , {I}^{L - 1} 〉\end{matrix} ] [ \begin{matrix}{v}_{1} \\ {v}_{2} \\ ⋮ \\ {v}_{L - 1}\end{matrix} ]\end{matrix}$$

Then, the linear mapping from the coordinate vector ν to the coordinate vector i is nonsingular, and described by
i=Bν
where, B is a (L-1)×(L-1) nonsingular matrix.

$$B = [ \begin{matrix}〈 {T}^{1} , {I}^{1} 〉 & 〈 {T}^{1} , {I}^{2} 〉 & ⋯ & 〈 {T}^{1} , {I}^{L - 1} 〉 \\ 〈 {T}^{2} , {I}^{1} 〉 & 〈 {T}^{2} , {I}^{2} 〉 & ⋯ & 〈 {T}^{2} , {I}^{L - 1} 〉 \\ ⋮ & ⋮ & ⋮ & ⋮ \\ 〈 {T}^{L - 1} , {I}^{1} 〉 & 〈 {T}^{L - 1} , {I}^{2} 〉 & ⋯ & 〈 {T}^{L - 1} , {I}^{L - 1} 〉\end{matrix} ] \qquad ( 2 )$$

Algorithm 1

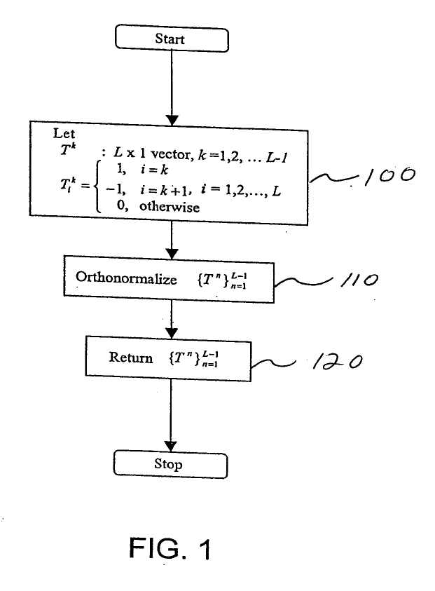

According to the present invention, an orthonormal basis set {Tⁿ})ₙ₌₁^{L-1} is first generated by algorithm 1. FIG. 1 shows how algorithm 1 of the present invention is carried out. The sum of the electrode currents through the body is zero, and likewise, the sum of the basis vector elements T^{k} must be zero. As shown in a first step 100 in FIG. 1, let T^{k}: L×1 vector, k=1,2, . . . L-1

$${T}_{i}^{k} = \{ \begin{matrix}1 , & i = k \\ - 1 , & i = k + 1 , i = 1 , 2 , … , L \\ 0 , & otherwise\end{matrix}$$

The vectors of the matrix are orthonormalized in step 110 and the orthonormal basis set {Tⁿ}ₙ₌₁^{L-1} is generated in step 120. A matrix is made orthogonal and normal by orthonormalization. In an orthogonal matrix, all column (or row) vectors are orthogonal to each other. In a normal matrix, each column (or row) vector has a unit norm. Hence, the basis vectors T^{k}, k=1, . . . , L-1 are orthogonal to each other, and each of the basis vector T^{k} has a unit norm.

Algorithm 2

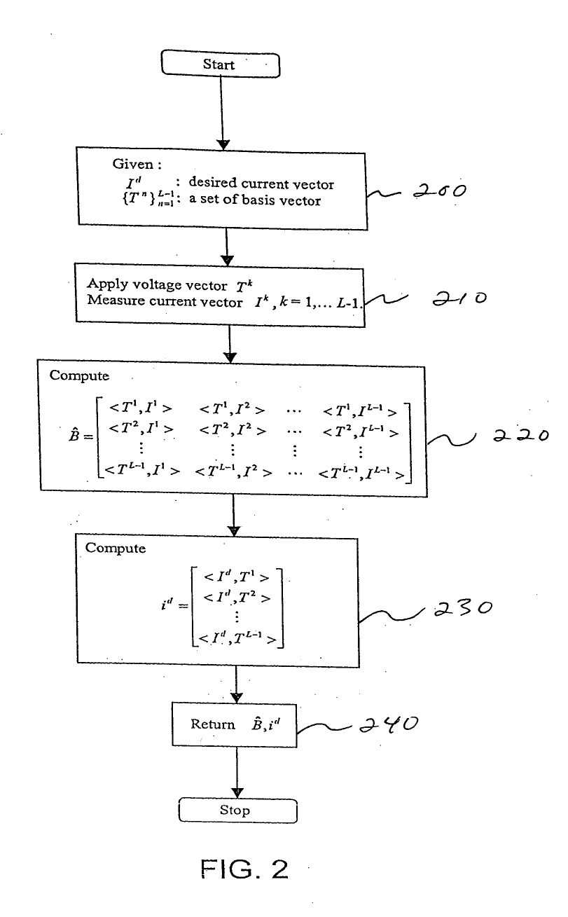

Turning to FIG. 2, given a desired current I^{d}, a basis set {Tⁿ}ₙ₌₁^{L-1} and the relationship from voltage coordinate vector to current coordinate vector i=Bν in a first step 200, an estimate of B denoted as \hat{B} is sought. In step 210, apply voltage T^{k} and measure I^{k}, k=1, . . . L-1. In step 220, compute \hat{B}

$$\hat{B} = [ \begin{matrix}〈 {T}^{1} , {I}^{1} 〉 & 〈 {T}^{1} , {I}^{2} 〉 & ⋯ & 〈 {T}^{1} , {I}^{L - 1} 〉 \\ 〈 {T}^{2} , {I}^{1} 〉 & 〈 {T}^{2} , {I}^{2} 〉 & ⋯ & 〈 {T}^{2} , {I}^{L - 1} 〉 \\ ⋮ & ⋮ & ⋮ & ⋮ \\ 〈 {T}^{L - 1} , {I}^{1} 〉 & 〈 {T}^{L - 1} , {I}^{2} 〉 & ⋯ & 〈 {T}^{L - 1} , {I}^{L - 1} 〉\end{matrix} ]$$

In step 230, compute i^{d}

$${i}^{d} = [ \begin{matrix}{i}_{1}^{d} \\ {i}_{2}^{d} \\ ⋮ \\ {i}_{L - 1}^{d}\end{matrix} ] = [ \begin{matrix}〈 {I}^{d} , {T}^{1} 〉 \\ 〈 {I}^{d} , {T}^{2} 〉 \\ ⋮ \\ 〈 {I}^{d} , {T}^{L - 1} 〉\end{matrix} ]$$

In step 240, \hat{B} and i^{d} are generated. Now the nonsingular mapping B, i=Bν can be used, in the exemplary algorithm above, and the procedure is summarized below.

Algorithm 3

Given a desired current I^{d}, and error tolerance ε, the goal is to find the voltage V* that will result in the current I* such that ∥e∥=∥I^{d}−I*∥<ε
1. Let e₀=i^{d}, ν⁰=V⁰=0, k=0
2. k=k+1. Compute ν^{k}=ν^{k-1}+\hat{B}⁻¹eₖ₋₁.
Apply

$${V}^{k} = {\sum }_{n = 1}^{L - 1} {v}_{n}^{k} {T}^{n} ,$$

and measure I^{k}.
Compute

$${i}^{k} = [ \begin{matrix}{i}_{1}^{k} \\ {i}_{2}^{k} \\ ⋮ \\ {i}_{L - 1}^{k}\end{matrix} ] = [ \begin{matrix}〈 {I}^{k} , {T}^{1} 〉 \\ 〈 {I}^{k} , {T}^{2} 〉 \\ ⋮ \\ 〈 {I}^{k} , {T}^{L - 1} 〉\end{matrix} ]$$
Compute eₖ=i^{d}−i^{k}
3. If ∥eₖ∥<ε, then V*=V^{k} and stop. Else go to 2 Note that in Algorithm 3, the mapping i=Bν is used in place of the initial mapping I=AV used in the exemplary algorithm.

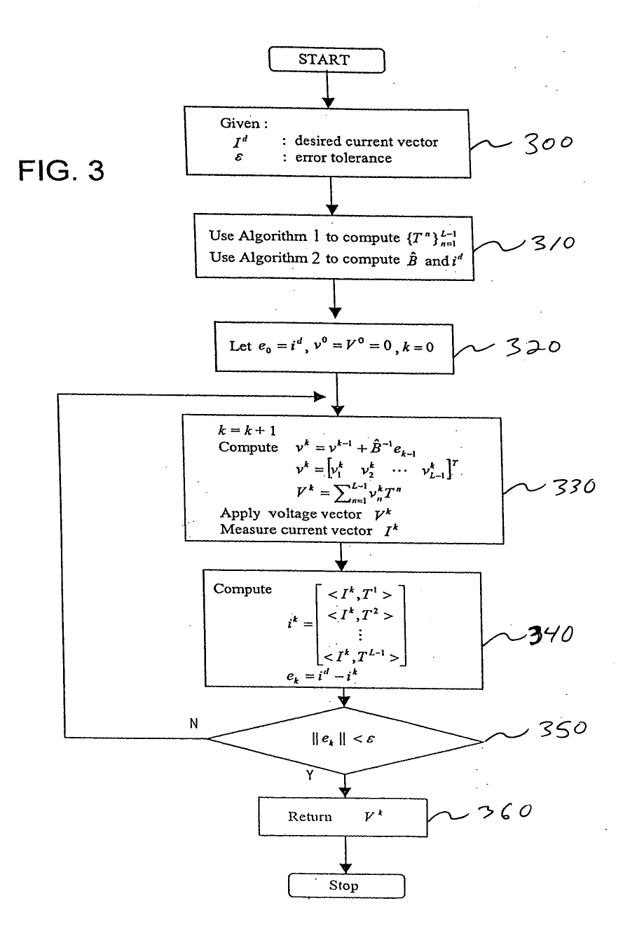

Algorithms 1, 2, and 3 are used to calculate a voltage that will generate a desired electrode current I^{d} in an EIT system. An overview flowchart for calculating the voltage is provided in FIG. 3.

In step 300, a desired current I^{d} and error tolerance ε are given. In step 310, use Algorithm 1 to compute a basis set {Tⁿ}ₙ₌₁^{L-1} and use Algorithm 2 to compute \hat{B}, i^{d}. In step 320, the following is defined.
let e₀=i^{d}, ν⁰=V⁰=0, k=0
The next set of steps are part of Algorithm 3 above. In step 330, compute ν^{k}=ν^{k-1}+\hat{B}⁻¹eₖ₋₁, apply

$${V}^{k} = {\sum }_{n = 1}^{L - 1} {v}_{n}^{k} {T}^{n} ,$$

and measure I^{k}. In step 340, compute

$${i}^{k} = [ \begin{matrix}{i}_{1}^{k} \\ {i}_{2}^{k} \\ ⋮ \\ {i}_{L - 1}^{k}\end{matrix} ] = [ \begin{matrix}〈 {I}^{k} , {T}^{1} 〉 \\ 〈 {I}^{k} , {T}^{2} 〉 \\ ⋮ \\ 〈 {I}^{k} , {T}^{L - 1} 〉\end{matrix} ]$$

and eₖ=i^{d}−i^{k} In step 350, determine whether ∥e_{k∥<ε}. If ∥eₖ∥<ε then V*=V^{k} in step 360 and stop. Else go to step 330. Note that in Algorithm 3, the mapping i=Bν is used in place of the initial mapping I=AV used in the exemplary algorithm above.

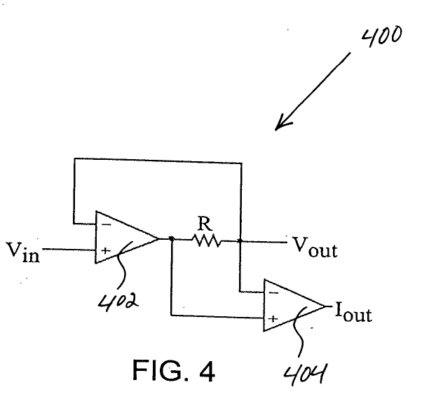

The EIT system of the present invention operates as follows. Algorithms 1, 2, and 3 defined above, are algorithms of the present invention that are used to calculate a voltage that will generate a desired electrode current I^{d} in an EIT system. The EIT system includes a plurality of voltage sources, such as the one shown in FIG. 4, which are used to produce or carry out the calculated voltage that will generate the desired electrode current I^{d} in the EIT system. FIG. 4 shows a voltage source 400, which provides a voltage Vᵢₙ at an operational amplifier 402 and a measuring circuit which is the combination of a resistor R and the operational amplifier 404. After the voltage Vᵢₙ is provided, Vₒᵤₜ and a signal Iₒᵤₜ are produced. The signal Iₒᵤₜ is a measure of the current that is going to the load while Vₒᵤₜ is produced.

In the EIT system of the present invention, a plurality of voltage sources 400 produce a plurality of voltage patterns. These voltage patterns are based on the calculated voltage which is determined by algorithms 1, 2, and 3 of the present invention. These calculated voltage patterns are applied to electrodes to create resulting current patterns in the body. The resulting current patterns are measured at the electrodes via the measuring circuit of voltage source 400 to determine at least one of conductivity and permittivity distributions within the body.

The algorithms 1, 2, and 3 of the present invention are used as follows to provide the calculated voltage that will generate a desired electrode current I^{d} in the EIT system.

After selecting a desired current vector (I^{d}) and an error tolerance (ε), algorithm 1 is used to compute an orthonormal basis set

$${\{ {T}^{n} \}}_{n = 1}^{L - 1} ,$$

and algorithm 2 with the orthonormal basis set and the desired current vector I^{d}, is used to compute an estimate of a non-singular linear mapping matrix for converting coordinate vector for voltage vector with respect to the orthonormal basis set to coordinate vector for current vector with respect to the orthonormal basis set and to compute coordinate vector for the desired current vector (i^{d}).

According to exemplary algorithm 3, the voltage of the voltage vector V^{k} is computed as a function of the estimate of the non-singular linear mapping matrix and the coordinate vector for the desired current vector. The voltages of the voltage vector V^{k} are applied to the electrodes of the EIT system. The resulting current vector is measured by the measuring circuit of the voltage sources 400. The coordinate vector i^{k} for the measured resulting current vector is computed with respect to the orthonormal basis set

$${\{ {T}^{n} \}}_{n = 1}^{L - 1} .$$

Finally, a calculation is made for a norm ∥eₖ∥ of the actual error between the coordinate vector i^{k} for the measured resulting current vector and the coordinate vector i^{d} for the desired current vector (e.g., eₖ=i^{d}−i^{k}).

If the norm ∥eₖ∥ of the actual error is less than the selected error tolerance ε, a voltage pattern is applied to the electrodes based on the voltage vector V^{k} that was computed in step 330 of algorithm 3.

If the norm ∥eₖ∥ of the actual error is greater than the selected error tolerance ε, then the third algorithm must be repeated. That is, the following steps are repeated. The voltage of the voltage vector V^{k} is computed as a function of the estimate of the non-singular linear mapping matrix and the coordinate vector for the desired current vector. The voltages of the voltage vector V^{k} are applied to the electrodes of the EIT system. The resulting current vector is measured by the measuring circuit of the voltage sources 400. The coordinate vector i^{k} for the measured resulting current vector is computed with respect to the orthonormal basis set

$${\{ {T}^{n} \}}_{n = 1}^{L - 1} .$$

Again, a norm ∥eₖ∥ is calculated of the actual error between the coordinate vector i^{k} for the measured resulting current vector and the coordinate vector i^{d} for the desired current vector (e.g., eₖ=i^{d}−i^{k}).

Simulation

The goal of the simulation was to examine the convergence of the current output to the desired value, and the effect of the estimation error of \hat{B} on the convergence using MATLAB. The test data were obtained from measurement data of a 2-D circular homogeneous saline phantom tank using ACT 3 ([5] P. M. Edic, G. J. Saulnier, J. C. Newell, D. Isaacson, “A real-time electrical impedance tomograph,” IEEE Trans. on Biomedical Eng., vol.42, no.9, pp.849-859, September 1995). The basis used for this circular 2D geometry is

$${T}_{l}^{n} = \{ \begin{matrix}{M}_{n} cos n {θ}_{l} , & \begin{matrix}n = 1 , 2 , ⋯ , \frac{L}{2} , & l = 1 , 2 , ⋯ , L\end{matrix} \\ {M}_{n} sin ( n - \frac{L}{2} ) {θ}_{l} , & \begin{matrix}n = \frac{L}{2} + 1 , ⋯ , L - 1 , & l = 1 , 2 , ⋯ , L\end{matrix}\end{matrix}$$

where θ, is the angle of the electrode I with respect to the center of the disk. Mₙ is chosen to normalize Tⁿ. In ACT 3, the number of electrodes is L=32. A total of 31 voltages resulting from 31 linearly independent current patterns were measured, and converted to their coordinate vectors. The matrix B was computed from (1), and regarded as the true mapping for i=Bv.

In order to simulate the estimation error, random multiplicative errors and additive errors were added to each element of B to make up \hat{B}. For example, to introduce 1% multiplicative error, a random number x was generated with uniform distribution between −0.01 and +0.01, and (1+x) was multiplied to each element of B. For additive error, xBₘₐₓ was added to each element of B, where Bₘₐₓ is the element of B with maximum absolute value. In order to simulate the current measurement noise, a set of random numbers was generated with uniform distribution between −1 and 1, the magnitude were adjusted so that the SNR is 105 dB (as reported in [3] R. D. Cook, G. J. Saulnier, D. G. Gisser, J. C. Goble, J. C. Newell, and D. Isaacson, “ACT 3: A high speed, high precision electrical impedance tomography,” IEEE Trans. on Biomedical Eng., vol.41, pp.713-722, August 1994), and were added to the currents.

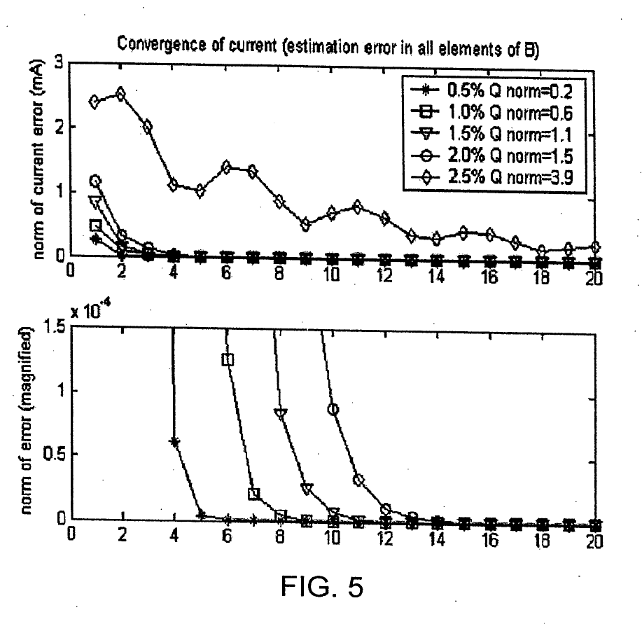

The desired current value used in the simulation was Iₖ^{d}=0.2 cos θₖ+j0.1 sin θₖ (mA) for the k-th electrode. The real part of I^{d} is one of the actual current patterns used in the ACT 3 measurements. The imaginary part was added for test purposes. FIG. 5 shows the convergence of the current as the iteration count increases. Five lines represent the results with different multiplicative and additive errors. For example, error 1.0% means that the multiplicative error of 1% and additive error of 1% were introduced as the estimation error. The lower figure shows the magnified portion of the upper figure. Note that the resolution of the 16 bit ADC is 1/2¹⁶=1.5×10⁻⁵, and the errors decrease below this value after 5˜12 iterations as shown in FIG. 5.

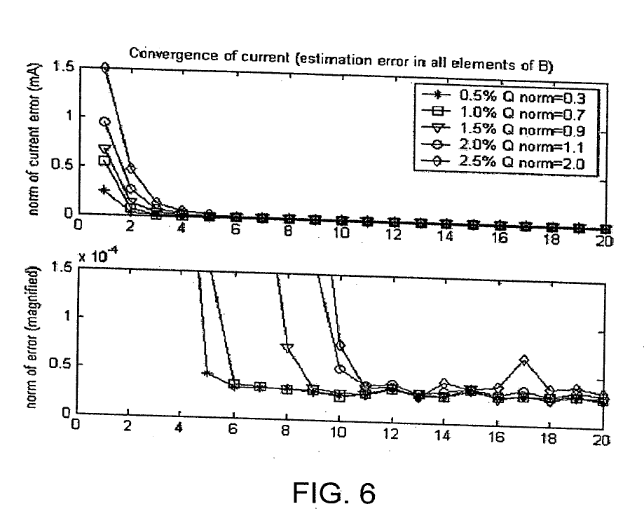

Also note that Theorem 1 implies that if the initial error ∥e₀∥₂=1 and ∥Q∥₂=0.1, it will take at most k=5 iterations to reduce the error below the resolution of the 16 bit ADC. It can be seen that when the estimation errors are 1.5%, 2.0%, and 2.5%, ∥Q∥₂ are greater than 1, but the current still converges to the desired value. This is because the convergence condition ∥Q∥₂<1 is a sufficient condition. Even when it is not satisfied, the current convergence is still possible, though not guaranteed. FIG. 6 shows the same simulation with the current measurement error added. It is seen that the current almost converges to the desired value, within the error bounds set by the noise. The remaining error is the consequence of the measurement noise.

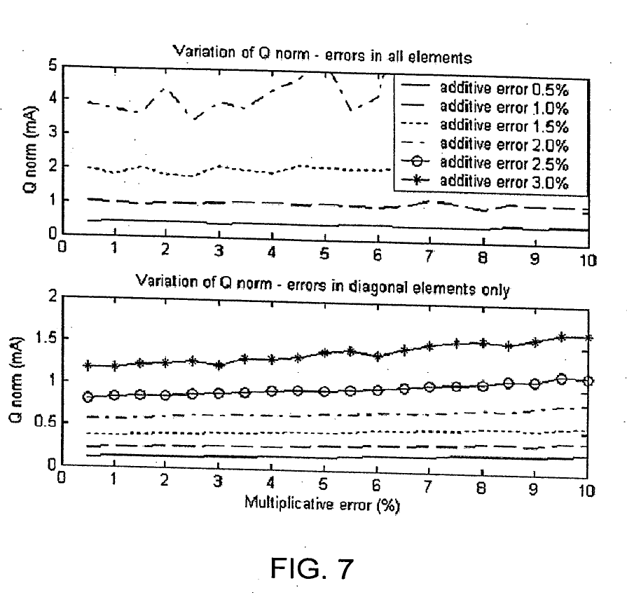

The speed of convergence and whether the current will converge at all depend on the magnitude of the estimation error in the form of ∥Q∥=∥I−B\hat{B}⁻¹∥. If ∥Q∥<1, it is guaranteed to converge to the desired value by Theorem 1. The speed of the convergence depends on the magnitude of ∥Q∥. If ∥Q∥≧1, current may still converge as shown in FIGS. 5 and 6. The next question is how realistic the condition ∥Q∥<1 is in practice. FIG. 7 shows the behavior of ∥Q∥₂ with the variation of multiplicative and additive errors. Multiplicative error and additive errors were varied independently, and their effect on ∥Q∥₂ was studied. Since the errors were generated by random numbers, for each combination of multiplicative error and additive error, ∥Q∥₂ was computed 1000 times and the maximum value was used as the value of ∥Q∥₂. It can be seen from the upper figure that ∥Q∥₂<1 when additive error was less than 1%. The multiplicative error had less significant influence because B was a diagonal matrix and the off-diagonal elements were zero. Since B is diagonal, we can force the off-diagonal elements of B to be zero, and apply estimation errors to the diagonal elements only. In this case, it can be seen from the lower figure that ∥Q∥₂<1 when additive error was less than 2.5%. This suggests that the knowledge of the true form of B can be used to reduce the effect of the estimation error.

It was shown that if the linear mapping from the voltage coordinate vector to the current coordinate vector can be estimated within a certain error bound, the current output produced by applying the voltage can be made to approach the desired value asymptotically. It was seen that when the convergence condition ∥Q∥₂<1 was satisfied, the current output approached the desired value. Additive error of 2.5% with multiplicative error of 7% could be tolerated to maintain the condition ∥Q∥₂<1. In practice, however, since we can never know the true value of B but only have the estimate \hat{B}, it is not possible to determine the value of ∥Q∥₂. If the current converges to a value, it is an indirect indication that the condition ∥Q∥₂<1 may have been satisfied.

While a specific embodiment of the invention has been shown and described in detail to illustrate the application of the principles of the invention, it will be understood that the invention may be embodied otherwise without departing from such principles.

1. An electrical impedance tomography method for determining at least one of an electrical conductivity and an electrical permittivity distribution within a body from measurements made at a plurality of electrodes spaced on a surface of the body, the method comprising:
(a) providing a plurality of voltage sources for producing a plurality of voltage patterns that are each calculated using an iterative calculation process;
(b) applying the calculated voltage patterns to the electrodes to create resulting current patterns in the body; and
(c) measuring the resulting current patterns at the electrodes to determine at least one of the conductivity and permittivity distributions within the body;
(d) the calculation process comprising:
(i) selecting a desired current vector and an error tolerance;
(ii) using a first algorithm to compute an orthonormal basis set;
(iii) using a second algorithm with the orthonormal basis set and the desired current vector to compute an estimate of a non-singular linear mapping matrix for converting coordinate vector for voltage vector with respect to the orthonormal basis set to coordinate vector for current vector with respect to the orthonormal basis set and to compute coordinate vector for the desired current vector;
(iv) computing and applying to the electrodes, the voltages of the voltage vector as a function of the estimate of the non-singular linear mapping matrix and the coordinate vector for the desired current vector;
(v) measuring the resulting current vector;
(vi) computing the coordinate vector for the measured resulting current vector with respect to the orthonormal basis set;
(vii) calculating a norm of the actual error between the coordinate vector for the measured resulting current vector and the coordinate vector for the desired current vector; and
(viii) if the norm of the actual error is greater than the selected error tolerance, repeating steps (iv) to (viii), and if the norm of the actual error is less than the selected error tolerance, using the computed voltage vector of step (iv) as one of the calculated voltage patterns to perform step (b).

2. An electrical impedance tomography method according to claim 1, wherein the voltage source comprises a resistor and an operational amplifier as a measuring circuit for measuring a signal Iₒᵤₜ which is a measure of current that is fed to said electrodes.

3. An electrical impedance tomography method according to claim 1, wherein the first algorithm includes the steps of:
providing
let T^{k}: L×1 vector, K=1,2, . . . L-1
$${T}_{i}^{k} = \{ \begin{matrix}1 , & i = k \\ - 1 , & \begin{matrix}i = k + 1 , & i = 1 , 2 , … , L\end{matrix} \\ 0 , & otherwise\end{matrix}$$
orthonormalizing the vectors of the matrix; and
generating the orthonormal basis set
$${\{ {T}^{n} \}}_{n = 1}^{L - 1} .$$

4. An electrical impedance tomography method according to claim 1, wherein the second algorithm includes the steps of:
applying voltage T^{k} and measuring I^{k}, k=1, . . . L-1;
computing \hat{B} based on
$$\hat{B} = [ \begin{matrix}〈 {T}^{1} , {I}^{1} 〉 & 〈 {T}^{1} , {I}^{2} 〉 & ⋯ & 〈 {T}^{1} , {I}^{L - 1} 〉 \\ 〈 {T}^{2} , {I}^{1} 〉 & 〈 {T}^{2} , {I}^{2} 〉 & ⋯ & 〈 {T}^{2} , {I}^{L - 1} 〉 \\ ⋮ & ⋮ & ⋮ & ⋮ \\ 〈 {T}^{L - 1} , {I}^{1} 〉 & 〈 {T}^{L - 1} , {I}^{2} 〉 & ⋯ & 〈 {T}^{L - 1} , {I}^{L - 1} 〉\end{matrix} ]$$
computing i^{d}
$${i}^{d} = [ \begin{matrix}{i}_{1}^{d} \\ {i}_{2}^{d} \\ ⋮ \\ {i}_{L - 1}^{d}\end{matrix} ] = [ \begin{matrix}〈 {I}^{d} , {T}^{1} 〉 \\ 〈 {I}^{d} , {T}^{2} 〉 \\ ⋮ \\ 〈 {I}^{d} , {T}^{L - 1} 〉\end{matrix} ] ;$$
and
generating \hat{B} and i^{d}.

5. An electrical impedance tomography method according to claim 1, wherein the voltages of the voltage vector are computed as a function of the estimate of the non-singular linear mapping matrix and the coordinate vector for the desired current vector by computing ν^{k}=ν^{k-1}+\hat{B}⁻¹eₖ₋₁.

6. An electrical impedance tomography method according to claim 1, wherein the voltages of the voltage vector are applied as a function of the estimate of the non-singular linear mapping matrix and the coordinate vector for the desired current vector by applying
$${V}^{k} = {\sum }_{n = 1}^{L - 1} {v}_{n}^{k} {T}^{n} .$$

7. An electrical impedance tomography method according to claim 1, wherein the coordinate vector for the measured resulting current vector is computed with respect to the orthonormal basis set by computing
$${i}^{k} = [ \begin{matrix}{i}_{1}^{k} \\ {i}_{2}^{k} \\ ⋮ \\ {i}_{L - 1}^{k}\end{matrix} ] = [ \begin{matrix}〈 {I}^{k} , {T}^{1} 〉 \\ 〈 {I}^{k} , {T}^{2} 〉 \\ ⋮ \\ 〈 {I}^{k} , {T}^{L - 1} 〉\end{matrix} ] .$$

8. An electrical impedance tomography method according to claim 1, wherein a norm of the actual error between the coordinate vector for the measured resulting current vector and the coordinate vector for the desired current vector is calculated by computing eₖ=i^{d}−i^{k}.

9. A method for calculating the voltage that will generate a desired electrode current in an EIT system, comprising the steps of:
(a) selecting a desired current vector and an error tolerance;
(b) using a first algorithm to compute an orthonormal basis set;
(c) using a second algorithm with the orthonormal basis set and the desired current vector to compute an estimate of a non-singular linear mapping matrix for converting coordinate vector for voltage vector with respect to the orthonormal basis set to coordinate vector for current vector with respect to the orthonormal basis set and to compute coordinate vector for the desired current vector;
(d) using a third algorithm comprising the steps of:
(i) computing and applying to the electrodes, the voltages of the voltage vector as a function of the estimate of the non-singular linear mapping matrix and the coordinate vector for the desired current vector;
(ii) measuring the resulting current vector;
(iii) computing the coordinate vector for the measured resulting current vector with respect to the orthonormal basis set;
(iv) calculating a norm of the actual error between the coordinate vector for the measured resulting current vector and the coordinate vector for the desired current vector; and
(v) if the norm of the actual error is greater than the selected error tolerance, repeating steps (i) to (v), and if the norm of the actual error is less than the selected error tolerance, using the computed voltage vector of step (i) as a calculated voltage that will generate a desired electrode current.

10. An electrical impedance tomography method according to claim 9, wherein the first algorithm includes the steps of:
providing
let T^{k}: L×1 vector, k=1,2, . . . L-1
$${T}_{i}^{k} = \{ \begin{matrix}1 , & i = k \\ - 1 , & \begin{matrix}i = k + 1 , & i = 1 , 2 , … , L\end{matrix} \\ 0 , & otherwise\end{matrix}$$
orthonormalizing the vectors of the matrix; and
generating the orthonormal basis set
$${\{ {T}^{n} \}}_{n = 1}^{L - 1} .$$

11. An electrical impedance tomography method according to claim 9, wherein the second algorithm includes the steps of:
applying voltage T^{k} and measuring I^{k}, k=1, . . . L-1;
computing \hat{B} based on
$$\hat{B} = [ \begin{matrix}〈 {T}^{1} , {I}^{1} 〉 & 〈 {T}^{1} , {I}^{2} 〉 & ⋯ & 〈 {T}^{1} , {I}^{L - 1} 〉 \\ 〈 {T}^{2} , {I}^{1} 〉 & 〈 {T}^{2} , {I}^{2} 〉 & ⋯ & 〈 {T}^{2} , {I}^{L - 1} 〉 \\ ⋮ & ⋮ & ⋮ & ⋮ \\ 〈 {T}^{L - 1} , {I}^{1} 〉 & 〈 {T}^{L - 1} , {I}^{2} 〉 & ⋯ & 〈 {T}^{L - 1} , {I}^{L - 1} 〉\end{matrix} ]$$
computing i^{d}
$${i}^{d} = [ \begin{matrix}{i}_{1}^{d} \\ {i}_{2}^{d} \\ ⋮ \\ {i}_{L - 1}^{d}\end{matrix} ] = [ \begin{matrix}〈 {I}^{d} , {T}^{1} 〉 \\ 〈 {I}^{d} , {T}^{2} 〉 \\ ⋮ \\ 〈 {I}^{d} , {T}^{L - 1} 〉\end{matrix} ] ;$$
generating \hat{B} and i^{d}.

12. An electrical impedance tomography method according to claim 9, wherein the voltages of the voltage vector are computed as a function of the estimate of the non-singular linear mapping matrix and the coordinate vector for the desired current vector by computing ν^{k}=ν^{k-1}+\hat{B}⁻¹eₖ₋₁.

13. An electrical impedance tomography method according to claim 9, wherein the voltages of the voltage vector are applied as a function of the estimate of the non-singular linear mapping matrix and the coordinate vector for the desired current vector by applying
$${V}^{k} = {\sum }_{n = 1}^{L - 1} {v}_{n}^{k} {T}^{n} .$$

14. An electrical impedance tomography method according to claim 9, wherein the coordinate vector for the measured resulting current vector is computed with respect to the orthonormal basis set by computing
$${i}^{k} = [ \begin{matrix}{i}_{1}^{k} \\ {i}_{2}^{k} \\ ⋮ \\ {i}_{L - 1}^{k}\end{matrix} ] = [ \begin{matrix}< {I}^{k} , {T}^{1} > \\ < {I}^{k} , {T}^{2} > \\ ⋮ \\ < {I}^{k} , {T}^{L - 1} >\end{matrix} ] .$$

15. An electrical impedance tomography method according to claim 9, wherein a norm of the actual error between the coordinate vector for the measured resulting current vector and the coordinate vector for the desired current vector is calculated by computing eₖ=i^{d}−i^{k}.

16. A method for calculating the voltage that will generate a desired electrode current in an EIT system, comprising the steps of:
(a) selecting a desired current vector and an error tolerance;
(b) using a first algorithm to compute an orthonormal basis set by providing
let T^{k}: L×1 vector, k=1,2, . . . L-1
$${i}^{d} {T}_{i}^{k} = \{ \begin{matrix}1 , i = k \\ - 1 , i = k + 1 , i = 1 , 2 , … , L \\ 0 , otherwise\end{matrix}$$
orthonormalizing the vectors of the matrix and generating the orthonormal basis set {Tⁿ}ₙ₌₁^{L-1};
(c) using a second algorithm which comprises applying voltage T^{k} and measuring I^{k}, k=1, . . . L-1;
computing \hat{B} based on
$$\hat{B} = [ \begin{matrix}< {T}^{1} , {I}^{1} > & < {T}^{1} , {I}^{2} > & … & < {T}^{1} , {I}^{L - 1} > \\ < {T}^{2} , {I}^{1} > & < {T}^{2} , {I}^{2} > & … & < {T}^{2} , {I}^{L - 1} > \\ ⋮ & ⋮ & ⋮ & ⋮ \\ < {T}^{L - 1} , {I}^{1} > & < {T}^{L - 1} , {I}^{2} > & … & < {T}^{L - 1} , {I}^{L - 1} >\end{matrix} ]$$
computing i^{d}
$${i}^{d} = [ \begin{matrix}{i}_{1}^{d} \\ {i}_{2}^{d} \\ ⋮ \\ {i}_{L - 1}^{d}\end{matrix} ] = [ \begin{matrix}< {I}^{d} , {T}^{1} > \\ < {I}^{d} , {T}^{2} > \\ ⋮ \\ < {I}^{d} , {T}^{L - 1} >\end{matrix} ]$$
and generating \hat{B} and i^{d};
(d) using a third algorithm comprising the steps of:
(i) computing and applying the voltages of the voltage vector by computing ν^{k}=ν^{k-1}+\hat{B}⁻¹eₖ₋₁ and applying
$${V}^{k} = {\sum }_{n = 1}^{L - 1} {v}_{n}^{k} {T}^{n} ;$$
(ii) measuring the resulting current vector;
(iii) computing the coordinate vector for the measured resulting current vector with respect to the orthonormal basis set by computing
$${i}^{k} = [ \begin{matrix}{i}_{1}^{k} \\ {i}_{2}^{k} \\ ⋮ \\ {i}_{L - 1}^{k}\end{matrix} ] = [ \begin{matrix}< {I}^{k} , {T}^{1} > \\ < {I}^{k} , {T}^{2} > \\ ⋮ \\ < {I}^{k} , {T}^{L - 1} >\end{matrix} ] ;$$
(iv) calculating a norm of the actual error between the coordinate vector for the measured resulting current vector and the coordinate vector for the desired current vector by computing eₖ=i^{d}−i^{k}; and
(v) if the norm of the actual error is greater than the selected error tolerance, repeating steps (i) to (v), and if the norm of the actual error is less than the selected error tolerance, using the computed voltage vector of step (i) as a calculated voltage that will generate a desired electrode current.
